(19) United States
(12) Patent Application Publication
(10) Pub. No.: US 2022/0062333 A1
(43) Pub. Date: Mar. 3, 2022

(54) Skin Care Formulation for the Treatment of Acne

(71) Applicant: BOTTLED SCIENCE LIMITED, London (GB)

(72) Inventor: Piers RAPER, Richmond (GB)

(21) Appl. No.: 17/465,415

(22) Filed: Sep. 2, 2021

Related U.S. Application Data
(60) Provisional application No. 63/074,097, filed on Sep. 3, 2020.

(51) Int. Cl. A61K 33/30; A61K 9/14; A61K 9/00; A61K 45/06; A61K 33/06; A61K 47/46; A61K 9/48; A61P 17/10

(52) U.S. Cl. CPC A61K 33/30; A61K 9/14; A61K 9/0095; A61K 45/06; A61P 17/10; A61K 47/46; A61K 9/4808; A61K 9/4833; A61K 33/06

(57) ABSTRACT

The present invention relates to a skin care formulation that is suitable for the treatment of acne and breakouts.

### CROSS-REFERENCE TO RELATED APPLICATION

This application claims benefit of U.S. Provisional Application No. 63/074097 filed Sep. 3, 2020, the complete disclosure of the prior application is hereby incorporated herein by reference in its entirety for all purposes.

### FIELD OF INVENTION

The present invention relates to a skin care formulation that is particularly useful in the treatment of acne and breakouts.

### BACKGROUND OF THE INVENTION

Acne is one of the most common skin disorders that affects both men and women not just in their teens but also later in life. Acne causes spots, oily skin and sometimes skin that is hot or painful to touch. It can have a major impact on an individual's self-image and confidence.

Acne and breakouts (which may or may not be caused by acne) form particularly on the face, neck, chest and back and are caused and exacerbated by a number of factors including hormone imbalance, bacteria, excess oil production, a diet high in sugar, blocked pores, poor digestive health, poor immune system and psychological factors (such as stress and anxiety). Reduced skin elasticity and poor hydration can also contribute to acne as they can result in dry and cracked skin which can facilitate the spread of bacteria.

The skin comprises sebaceous glands that are attached to hair follicles. Sebaceous glands secrete sebum, an oily substance which lubricates the hair and skin to stop it drying out. In acne, the sebaceous glands begin to produce too much sebum. The excess sebum mixes with dead skin cells and both substances (i.e. the excess sebum and dead skin cells) form a plug in the follicle. If the plugged follicle is close to the surface of the skin, it bulges outwards, creating a whitehead. Alternatively, the plugged follicle can be open to the skin, creating a blackhead. Normally harmless bacteria that live on the skin can then contaminate and infect the plugged follicles, causing papules, pustules, nodules or cysts. Cystic acne is a serious type of acne and develops when cysts form deep underneath the skin.

Several topical gels and creams that allege to treat acne and breakouts are known and are commercially available such as Proactiv, Epiduo Forte and Murad Clear Control. These tend to use adapalene (a retinoid), benzoyl peroxide or a combination thereof. Although these gels and creams may have an effect on mild or moderate acne, they are ineffective against severe cases, e.g. cystic acne. Several cleansers and creams are aimed at this problem, however, these contain little more than salicylic acid or an antibacterial cleaning agent, which are ineffective in the treatment of cystic acne.

Acne can also be treated medically using strong antibiotics such as doxycycline. However, antibiotics only address the bacterial causes of acne. Oral contraceptives (such as Ortho Tri-Cyclen) and anti-androgens (such as spironolactone) are routinely prescribed to treat acne, however, these only address the hormonal causes of acne. None of these treatments offer a complete solution for treating acne as they only address one aspect of the problem.

Isotretinoin (sold under the brand name Roaccutane) offers a treatment of severe acne. However, treatment with isotretinoin can reportedly have serious side effects. Thus, treatment with isotretinoin is avoided where possible.

There is a need for an effective treatment of acne (particularly cystic acne) and breakouts without the negative side effects associated with known medical treatments. The present invention addresses this need. Specifically, the present invention provides a skin care formulation that is administered orally (preferably as a beverage and a capsule) and thus works from the inside of the body to regulate hormonal imbalances, reduce inflammation, regulate excess oil production, detoxify the dermal layers of the skin, boost the immune system, improve skin elasticity and hydration, and improve digestive health. Accordingly, the present invention is particularly useful in the treatment of acne and breakouts. The present invention is advantageous over topical creams and gels as it works from the inside and is therefore able to support skin all over the body. Accordingly, the present invention is effective whether the acne is on the face, back, chest or neck. Furthermore, the skin care formulation according to the present invention does not cause the negative side effects associated with medical treatments of acne.

### SUMMARY OF INVENTION

In a first aspect, the present invention provides a skin care formulation comprising:
(i) one or more compound(s) that are capable of reducing estrogen concentration in a subject;
(ii) one or more compound(s) that are capable of reducing dihydrotestosterone concentration in a subject; and
(iii) one or more compound(s) that are capable of reducing insulin concentration in a subject.

In a preferred first aspect, the skin care formulation is in the form of a liquid composition and a solid composition. In a further preferred first aspect, the liquid composition is a beverage and the solid composition is a capsule.

In a second aspect, the present invention provides a method of treating acne comprising administering an effective amount of a skin care formulation according to the first aspect of the present invention to a subject in need thereof. In a preferred second aspect, the formulation is in the form of a solid composition and a liquid composition and the solid composition and liquid composition are administered concurrently or sequentially.

The present invention also provides a solid composition for oral administration comprising:
(i) one or more compound(s) that are capable of reducing estrogen concentration in a subject; and
(ii) one or more compound(s) that are capable of reducing dihydrotestosterone concentration in a subject.

The present invention also provides a liquid composition for a skin care beverage comprising:
(i) one or more compound(s) that are capable of reducing estrogen concentration in a subject;
(ii) one or more compound(s) that are capable of reducing dihydrotestosterone concentration in a subject; and
(iii) one or more compound(s) that are capable of reducing insulin concentration in a subject.

The present invention also provides a skin care beverage comprising the liquid composition provided hereinabove and a liquid diluent. Preferably, the liquid diluent is water.

The present invention also provides a method of preparing a skin care beverage provided hereinabove, wherein the method comprises mixing the liquid composition provided hereinabove and a liquid diluent. Preferably, the liquid diluent is water.

The present invention also provides a method of treating acne comprising administering an effective amount of a skin care beverage provided hereinabove or prepared as provided hereinabove and an effective amount of a solid composition provided hereinabove to a subject in need thereof.

The present invention also provides a skin care product comprising:
(i) a solid composition for oral administration as provided hereinabove; and
(ii) a liquid composition for a skin care beverage as provided hereinabove.

The present invention also provides use of the solid composition for oral administration provided hereinabove for treating acne.

The present invention also provides use of the liquid composition provided hereinabove for treating acne.

The present invention also provides use of the skin care beverage provided hereinabove or prepared as provided hereinabove for treating acne.

The present invention also provides use of the skin care product provided hereinabove for treating acne.

The present invention also provides a method of treating acne comprising administering an effective amount of the solid composition provided hereinabove to a subject in need thereof.

The present invention also provides a method of treating acne comprising administering an effective amount of the liquid composition provided hereinabove to a subject in need thereof.

The present invention also provides a method of treating acne comprising administering an effective amount of the skin care beverage provided hereinabove or prepared as provided hereinabove to a subject in need thereof.

The present invention also provides a method of treating acne comprising administering an effective amount of the skin care product provided hereinabove to a subject in need thereof.

### BRIEF DESCRIPTION OF FIGURES

FIG. 1 shows an area of an individual's skin affected by acne before (left-hand side) and after (right-hand side) 30 days of treatment according to the present invention.

FIG. 2 shows an area of an individual's skin affected by acne before (left-hand side) and after (right-hand side) 90 days of treatment according to the present invention.

FIGS. 3A-F show an area of an individual's skin affected by severe acne before (left-hand side) and after (right-hand side) 30 days of treatment according to the present invention.

The patent or application file contains at least one drawing executed in color. Copies of this patent or patent application publication with color drawing(s) will be provided by the Office upon request and payment of the necessary fee.

### DETAILED DESCRIPTION

Hereinafter, the present invention will be described in further detail with reference to suitably preferred embodiments. Any combination of the preferred ingredients (or compounds) and amounts herein may be present in the formulation, and the scope of the invention is not limited to the specific combinations and amounts of ingredients disclosed.

Skin Care Formulation

The present invention provides a skin care formulation comprising (i) one or more compound(s) that are capable of reducing estrogen concentration in a subject; (ii) one or more compound(s) that are capable of reducing dihydrotestosterone concentration in a subject; and (iii) one or more compound(s) that are capable of reducing insulin concentration in a subject. Preferably, the subject is a human.

As used herein, the term “reducing estrogen” typically refers to reducing an excess of estrogen or “rebalancing” estrogen levels (i.e. concentration) in a subject. An excess of estrogen is associated with a breakout or acne in a subject. Accordingly, a breakout or acne in a subject is indicative of an excess of estrogen. Once the breakout or acne are no longer present in a subject this indicates that the estrogen concentration is “balanced”, i.e. it is no longer in excess.

The skin care formulation according to the present invention may be used in a method of treating breakouts or acne, preferably cystic acne.

To be effective in the treatment of breakouts or acne all of the ingredients (i.e. compounds) in the formulation according to the present invention have to be absorbed by the body at the correct speed and concentration. If all of the ingredients were formulated in a solid (e.g. in a tablet or capsule) then a large number of tablets or capsules (e.g. about 30) would be required as it is not possible to formulate all of the ingredients in a single tablet or capsule due to ingredient stability and/or reactivity between ingredients. Taking a large number of tablets regularly (e.g. each day) is not desirable for a subject. In addition, if all of the ingredients were formulated in a solid then the amount of each ingredient in the solid would have to be at least about 3 times greater than the required dose of the ingredient because typically, when the ingredients are administered in solid form only about 35% of the active material would be absorbed by the body.

It is desirable for the ingredients to be formulated as a liquid that can be consumed orally, e.g. as a beverage, in order to enable the body to absorb all of the ingredients at the rate and concentration that provides efficacy. However, formulating all of the ingredients in a liquid has various drawbacks. For example, some ingredients are unstable in solution. Therefore, formulating these ingredients in a liquid would not provide a formulation that was effective in the treatment of breakouts or acne as the unstable ingredients would decompose, thereby losing effectiveness. Similarly, some ingredients may react with each other if they are in a liquid component. This would result in the loss of the active ingredients from the formulation and the formation of reaction products that may have no effect in the treatment of breakouts or acne, or that have a detrimental effect. Furthermore, some ingredients have a bitter taste and therefore including them in a liquid that is intended for oral administration (preferably as a beverage) results in a treatment that is not palatable to the subject.

The present inventors have solved the above mentioned problems by formulating the formulation according to the present invention as a solid composition and a liquid composition. Accordingly, in a preferred embodiment, the skin care formulation according to the present invention is in the form of a solid composition and a liquid composition. Preferably, the solid composition is a capsule, more preferably a slow release capsule. Preferably, the liquid composition is a beverage.

As used herein, the term “capsule” refers to a solid, wherein the active ingredients are enclosed in a hard or soft shell. Preferably, the shell comprises gelatin or cellulose, preferably hydroxypropyl methyl cellulose (HMPC). Preferably, the hydroxypropyl methyl cellulose is combined with titanium dioxide.

Preferably, the one or more compound(s) that are capable of reducing estrogen concentration is selected from indole-3-carbinol (I3C), a broccoli extract, an agnus castus extract, magnesium or a salt thereof, and combinations thereof. Preferably, the one or more compound(s) capable of reducing estrogen concentration is indole-3-carbinol.

Indole-3-carbinol (I3C) is derived from the enzymatic breakdown of glucobrassicin, a compound found in cruciferous vegetables such as broccoli, Brussels sprouts, cabbage, cauliflower, collard greens and kale. In the stomach, I3C molecules undergo add-catalysed condensation which generates a number of biologically active I3C oligomers such as 3,3′-diindolyirnethane (DIM). DIM is believed to be useful in the metabolism of estrogen and the metabolism of toxins. However, DIM is difficult for the body to digest and can lose it effectiveness upon digestion. The present invention circumvents digestion of this powerful compound by creating DIM in the body from I3C. The DIM then acts to reduce estrogen concentration. hi addition to being capable of reducing estrogen concentration in a subject, indole-3-carbinol may be involved in the metabolism of toxins. Accordingly, I3C is also capable of functioning as a detoxifying compound in the present invention.

Preferably, the indole-3-carbinol is present in the solid composition, preferably in an amount of from about 1 to about 20 wt %, preferably from about 3 to about 15 wt %, more preferably from about 5 to about 12 wt % of the solid composition.

Preferably, the one or more compound(s) capable of reducing estrogen concentration is a broccoli extract. Broccoli is a natural and powerful supply of the compound sulforaphane, which can function as a detoxifying compound, an anti-inflammatory and a hormone regulator. Preferably, the one or more compound(s) capable of reducing estrogen concentration is sulforaphane, preferably in the form of a broccoli extract.

Preferably, the broccoli extract used in the present invention is prepared by solvent extraction using a mixture of water and ethanol as the solvent to provide an extract that is 5% sulforaphane, Preferably, the solvent mixture used to provide the broccoli extract is a 50:50 mix, preferably a 70:30 mix, more preferably an 80:20 mix of water and ethanol respectively. Preferably, the broccoli extract is present in the solid composition, preferably in an amount of from about 0.5 to about 10 wt %, preferably from about 1 to about 8 wt %, more preferably from about 1.5 to about 6 wt % of the solid composition.

Without being bound in this regard, it is believed that the broccoli extract and/or sulforaphane facilitates the metabolism of indole-3-carbinol into 3,3′-diindolylmethane in the body. Therefore, the one or more compound(s) capable of reducing estrogen concentration preferably comprises indole-3-carbinol and a broccoli extract and/or sulforaphane. Preferably, the weight ratio of indole-3-carbinol to broccoli extract and/or sulforaphane is from about 3:1 to about 6:1, more preferably about 4:1.

Preferably, the one or more compound(s) that are capable of reducing estrogen concentration is agnus castus extract (also referred to as chasteberry extract). Preferably, the agnus castus extract used herein is prepared by solvent extraction using a mixture of water and ethanol. Preferably, the solvent mixture used is a 50:50 mix, preferably a 70:30 mix, more preferably an 80:20 mix of water and ethanol respectively. Without being bound in this regard, it is believed that the agnus castus extract delivers phytoprogesterones (e.g. apigenin) that balance excessive production of estrogens. Preferably, the agnus castus extract is present in the solid composition, preferably in an amount of from about 5 to about 35 wt %, preferably from about 10 to about 30 wt %, more preferably from about 15 to about 25 wt % of the solid composition. Preferably, the agnus castus extract is used in combination with indole-3-carbinol and/or a broccoli extract (optionally in the form of sulforaphane).

Preferably, the agnus castus extract is a source of apigenin. In a preferred embodiment, the one or more compound(s) capable of reducing estrogen concentration is apigenin, preferably in the form of an agnus castus extract.

Preferably, the one or more compound(s) that are capable of reducing estrogen concentration is a combination of indole-3-carbinol (I3C), broccoli extract (optionally in the form of sulforaphane) and agnus castus extract (optionally in the form of apigenin). Preferably, the weight ratio of I3C:broccoli extract:agnus castus extract is from about 2:1:4 to about 6:1:12, more preferably about 4:1:8.

Preferably, the one or more compound(s) that are capable of reducing estrogen concentration is magnesium or a salt thereof. Preferably, the one or more compound(s) that are capable of reducing estrogen concentration is magnesium citrate. Preferably, the magnesium or salt thereof is present in the liquid composition, preferably in an amount of from about 50 to about 500 mg/15 mL, preferably from about 100 to about 400 mg/15 mL, more preferably from about 150 to about 300 mg/15 mL of the composition.

Preferably, the one or more compound(s) capable of reducing estrogen concentration is a combination of indole-3-carbinol, broccoli extract (optionally in the form of sulforaphane), agnus castus extract (optionally in the form of apigenin) and magnesium (optionally in the form of magnesium citrate).

Preferably, the one or more compound(s) that are capable of reducing dihydrotestosterone (DHT) concentration is selected from saw palmetto, red reishi mushroom (also known as Ganoderma lucidum and lingzhi) extract, zinc or salts thereof, and combinations thereof. As used herein, saw palmetto refers to the extract from the berries of the saw palmetto tree. Preferably, the saw palmetto and red reishi mushroom extract are used in combination. Preferably, the weight ratio of saw palmetto to red reishi mushroom extract is from about 1:1 to about 1:5, more preferably about 1:2.

Without being bound in this regard, it is believed that in the present invention saw palmetto prevents DHT formation and red reishi mushroom extract lowers the formation of dihydrotestosterone from testosterone in the body by reducing levels of 5-alpha reductase, the enzyme that facilitates conversion of testosterone to dihydrotestosterone. Accordingly, this combination of compounds can reduce the concentration of dihydrotestosterone in the body. In addition, the red reishi mushroom extract may also boost the immune system.

Saw palmetto predominantly comprises fatty acids such as lauric acid, linoleic acid, oleic acid and myristic acid, which are also capable of inhibiting 5a-reductase and thus reducing the concentration of DHT. In addition, linoleic acid can function as an anti-inflammatory. Saw palmetto also comprises beta-sitosterol, which is also capable of reducing DHT concentration and reducing the number of follicles that contribute to acne. Preferably, the one or more compound(s) that are capable of reducing dihydrotestosterone (DHT) concentration is selected from lauric acid, linoleic acid, oleic acid, myristic acid, beta-sitosterol and combinations thereof, preferably in the form of saw palmetto.

Red reishi mushroom extract predominantly comprises polysaccharides and triterpenes as well as phenols, steroids, amino acids, lignin, vitamins and nucleosides. Without being bound in this regards, it is believed that it is the polysaccharide compounds (typically those with a linear (1→3)-β-d-Glcp as the main chain linkage) in red reishi mushroom extract that are responsible for the inhibition of 5-alpha reductase and thus reduction of DHT concentration.

Preferably, the saw palmetto is present in the solid composition, preferably in an amount of from about 5 to about 35 wt %, preferably from about 10 to about 30 wt %, more preferably from about 15 to about 25 wt % of the solid composition. Preferably, the red reishi mushroom extract is a powder extract. Preferably, the red reishi mushroom extract is present in the solid composition, preferably in an amount of from about 10 to about 60 wt %, preferably from about 20 to about 50 wt %, more preferably from about 30 to about 40 wt % of the solid composition. Preferably, the saw palmetto and red reishi mushroom extract used herein are prepared by solvent extraction using a mixture of water and ethanol. Preferably, the solvent mixture used is a 50:50 mix, preferably a 70:30 mix, more preferably an 80:20 nix of water and ethanol respectively.

Zinc is also an important compound in the metabolism of testosterone. Preferably, the one or more compound(s) that are capable of reducing dihydrotestosterone concentration is zinc or a salt thereof. Without being bound in this regard it is believed that zinc strengthens the immune system, helps control bacteria, contributes to tissue regeneration and controls inflammation as well as being key in the metabolism of testosterone to dihydrotestosterone. In addition, zinc is also believed to play a role in the absorption of vitamin A, regulate levels of vitamin E in the blood and contribute to enzymatic processes such as the manufacture of superoxide dismutase, which is an antioxidant. Hence, zinc can perform any of these additional functions in the present invention.

Preferably, the one or more compound(s) that are capable of reducing dihydrotestosterone concentration is zinc gluconate. Preferably, the zinc or salt thereof is present in the liquid composition, preferably in an amount of 5 to about 50 mg/15 mL, preferably from about 10 to about 40 mg/15 mL, more preferably from about 15 to about 30 mg/15 mL of the liquid composition.

Without being bound in this regard, it is believed that the combination of zinc (or salt thereof) and saw palmetto is capable of reducing the concentration of DHT to a greater extent than if each ingredient is used alone. Accordingly, the one or more compound(s) that are capable to reducing DHT concentration is a combination of zinc or a salt thereof and saw palmetto. Preferably, the one or more compound(s) that are capable to reducing DHT concentration is a combination of zinc or a salt thereof, saw palmetto and red reishi mushroom extract.

Preferably, the one or more compound(s) that are capable of reducing insulin concentration is chromium or a salt thereof. Advantageously, without being bound in this regard, it is believed that chromium increases glucose tolerance and improves insulin sensitivity in the body. Preferably, the chromium is chromium chloride or a hydrate thereof, more preferably the chromium is chromium chloride hexahydrate. Preferably, the chromium or salt thereof is present in the liquid composition of the skin care formulation, preferably in an amount of 5 to about 100 μg/15 mL, preferably from about 15 to about 80 μg/15 mL, more preferably from about 25 to about 80 μg/15 mL of the liquid composition.

Preferably, the skin care formulation of the present invention further comprises a digestive aid, preferably a probiotic. Preferably, the probiotic is lactobacillus. Preferably, the skin care formulation according to the present invention comprises about 2 billion cfu of lactobacillus. Preferably, the lactobacillus is present in the solid composition, preferably in an amount of from about 1 to about 25 wt %, preferably from about 5 to about 20 wt %, more preferably from about 10 to about 15 wt % of the total solid composition.

Preferably, the skin care formulation further comprises an omega-3 fatty acid. Preferably, the omega-3 fatty acid is from algal oil. Preferably, the omega-3 fatty acid is docosahexaenoic acid (DHA). Preferably, the omega-3 fatty acid is present in the liquid composition, preferably in an amount of from about 50 to about 500 mg/15 mL, preferably from about 100 to about 400 mg/15 mL, more preferably from about 150 to about 300 mg/15 mL of the liquid composition. Without being bound in this regard, it is believed that the omega-3 fatty acid hydrates the skin, regulates sebaceous activity and lowers inflammation.

Preferably, the skin care formulation further comprises vitamin A or a derivative thereof. Without being bound in this regard, it is believed that vitamin A or a derivative thereof lowers follicular hyperkeratinisation and excess sebum production. More specifically, it is believed that vitamin A or a derivative thereof lowers oil production and the build-up of keratin in skin follicles. Preferably, the vitamin A or derivative thereof is retinol acetate. Preferably, the vitamin A or derivative thereof is present in the liquid composition, preferably in an amount of from about 1 to about 50 mg/15 mL, preferably from about 3 to about 35 mg/15 mL, more preferably from about 5 to about 20 mg/15 mL of the liquid composition.

Preferably, the skin care formulation further comprises one or more additional detoxifying compound(s). Preferably, the one or more additional detoxifying compound(s) is milk thistle extract and/or methyl sulfonyl methane (MSM). Preferably, the one or more additional detoxifying compound(s) is milk thistle extract and methyl sulfonyl methane (MSM). Traditionally, milk thistle extract is made from the milk thistle seeds, which contain silymarin. Accordingly, the predominant compound in milk thistle extract is typically silymarin. Silymarin is a complex mixture of polyphenolic molecules, including seven closely related flavonolignans (silybin A, silybin B, isosilybin A, isosilybin B, silychristin, isosilychristin, silydianin) and one flavonoid (taxifolin). Preferably, the skin care formulation further comprises silybin A, silybin B, isosilybin A, isosilybin B, silychristin, isosilychristin, silydianin or taxifolin, preferably from a milk thistle extract. In an alternative preferred embodiment, the skin care formulation further comprises silybin A, silybin B, isosilybin A, isosilybin B, silychristin, isosilychristin, silydianin or taxifolin, wherein the flavonolignans or flavonoid are not from a milk thistle extract. As well as being a detoxifying compound, milk thistle extract can also function as an anti-inflammatory. Similarly, as well as being a detoxifying compound, MSM can function as an anti-inflammatory and improve cellular osmotic flow, which is key to elastin production.

Preferably, the milk thistle extract used herein is prepared by solvent extraction using a mixture of water and ethanol as the solvent to provide an extract that is 80% silymarin. Preferably, the solvent mixture used to provide the milk thistle extract is a 50:50 mix, preferably a 70:30 mix, more preferably an 80:20 mix of water and ethanol respectively. Preferably, the milk thistle extract is present in the liquid composition, preferably in an amount of from about 300 to about 900 mg/15 mL, preferably from about 400 to about 800 mg/15 mL, more preferably from about 500 to about 700 mg/15 mL of the liquid composition. Preferably, the MSM is present in the liquid composition, preferably in an amount of from about 200 to about 800 mg/15 mL, preferably from about 300 to about 700 mg/15 mL, more preferably from about 400 to about 600 mg/15 mL of the liquid composition.

Preferably, the skin care formulation according to the present invention further comprises selenium or a salt thereof. Without being bound in this regard, it is believed that selenium is important for skin elasticity, prevents inflammation and can be effective against oily skin. Preferably, the selenium is sodium selenium, more preferably the selenium is anhydrous sodium selenium. Preferably, the selenium is present in the liquid composition, preferably in an amount of from about 20 to about 200 μg/15 mL, preferably from about 50 to about 180 μg/15 mL, more preferably from about 80 to about 150 μg/15 mL of the liquid composition.

Preferably, the skin care formulation further comprises one or more antioxidant(s). Preferably, the one or more antioxidant(s) is L-cysteine or a derivative thereof. Preferably, the L-cysteine is N-acetyl-L-cysteine. Preferably, the N-acetyl-L-cysteine is present in the liquid composition, preferably in an amount of from about 100 to about 700 mg/15 mL, preferably from about 200 to about 600 mg/15 mL, more preferably from about 300 to about 500 mg/15 mL of the liquid composition.

Preferably, the skin care formulation further comprises vitamin C or a derivative thereof. Without being bound in this regard, vitamin C is believed to be an important component to new collagen formation. Preferably, the vitamin C derivative is vitamin C calcium ascorbate. Preferably, the vitamin C or derivative thereof is present in the liquid, preferably in an amount of from about 200 to about 800 mg/15 mL, preferably from about 300 to about 700 mg/15 mL, more preferably from about 400 to about 600 mg/15 mL of the liquid composition.

Preferably, the skin care formulation further comprises at least one vitamin B or derivate thereof. Preferably, the vitamin B is vitamin B1 or a derivative thereof (preferably thiamine hydrochloride), vitamin B2 or a derivative thereof (preferably riboflavin), vitamin B3 or a derivative thereof (preferably niacinamide), vitamin B5 or a derivative thereof (preferably calcium pantothenate), vitamin B6 or a derivative thereof (preferably pyridoxine hydrochloride), vitamin B7 or a derivative thereof (preferably biotin), vitamin B9 or a derivative thereof (preferably folic acid) and/or vitamin B12 or a derivative thereof (preferably cobalamin).

Preferably, the skin care formulation further comprises a vitamin B complex comprising vitamin B1 or a derivative thereof (preferably thiamine hydrochloride), vitamin B2 or a derivative thereof (preferably riboflavin), vitamin B3 or a derivative thereof (preferably niacinamide), vitamin B5 or a derivative thereof (preferably calcium pantothenate), vitamin B6 or a derivative thereof (preferably pyridoxine hydrochloride), vitamin B7 or a derivative thereof (preferably biotin), vitamin B9 or a derivative thereof (preferably folic acid) and/or vitamin B12 or a derivative thereof (preferably cobalamin). Preferably, the vitamin B complex is present in the liquid composition, preferably in an amount of from about 10 to about 300 mg/15 mL, preferably from about 25 to about 200 mg/15 mL, more preferably from about 50 to about 100 mg/15 mL of the liquid composition. Without being bound in this regard, it is believed that he vitamin B complex can promote more uniform, clearer, glowing and radiant skin. Furthermore, it is believed that the vitamin B6 or derivative thereof can play a role in the reduction of dihydrotestosterone.

Preferably, the skin care formulation further comprises calcareous marine algae or a derivative thereof. Calcareous marine algae can regulate the body's use of magnesium as well as functioning as a source of calcium. Preferably, the calcareous marine algae is present in the liquid composition, preferably in an amount of from about 100 to about 700 mg/15 mL, preferably from about 200 to about 600 mg/15 mL, more preferably from about 300 to about 500 mg/15 mL of the liquid composition.

Preferably, the skin care formulation according to the present invention further comprises calendula flower extract. Calendula flower extract comprises a variety of flavonoids and saponins that are believed to be key anti-inflammatory compounds. Preferably, the calendula flower extract is present in the liquid composition, preferably in an amount of from about 50 to about 600 mg/15 mL, preferably from about 100 to about 500 mg/15 mL, more preferably from about 150 to about 400 mg/15 mL of the liquid composition.

Preferably, the skin care formulation may further comprise one or more additional compound(s) that can improve insulin sensitivity and help control, preferably in an amount of from about 20 to about 600 mg/15 mL, preferably from about 40 to about 300 mg/15 mL, more preferably from about 80 to about 150 mg/15 mL of the liquid composition.

The skin care formulation according to the present invention may further comprise one or more additional ingredients that are useful in treating acne such as one or more further compound(s) useful for balancing hormones (e.g. estrogen and DHT), one or more further anti-inflammatory compound(s), one or more further detoxifying compound(s), one or more compound(s) that improve skin elasticity, one or more compound(s) that improve osmotic flow, one or more further compound(s) that strengthen or boost the immune system, one or more compound(s) that are effective against oily skin, one or more antioxidant compound(s), one or more compound(s) that promote collagen production, one or more compound(s) that are capable of increasing glucose tolerance, one or more compound(s) that hydrate the skin and/or one or more compound(s) that can regulate sebaceous activity.

As described above, the skin care formulation according to the present invention is preferably in the form of a solid composition and a liquid composition. Preferably, the solid composition is formulated as a capsule, more preferably a slow release capsule. Accordingly, the solid composition of the skin care formulation may further comprise one or more additional ingredients that are typically used in forming capsules for oral administration, for example, magnesium stearate. Typically, the capsule is prepared by mixing the ingredients of the solid composition in the required amount and then filling half of the capsule in a capsule filling machine before sealing the capsule with the second half of the capsule. Typically, the solid ingredients used to prepare the capsules are powders.

Preferably, the liquid composition of the skin care formulation according to the present invention further comprises a liquid diluent. Suitable liquid diluents are water, fruit juice and water-based flavoured beverages. Preferably, the liquid diluent is water. Preferably, the liquid diluent content of the liquid composition is at least about 80%, preferably at least about 85%, more preferably at least 90% of the total weight of the liquid composition. Preferably, the liquid composition is a beverage.

As used herein, there term “liquid composition” includes suspensions. Accordingly, the beverage may comprise microsolids suspended in solution.

Preferably, the liquid composition of the skin care formulation according to the present invention further comprises one or more additional components to enhance the liquid composition, for example one or more flavouring agent(s), colourant(s), sweetener(s), fruit juice concentrate and acidulant(s) may be included. Preferably, a flavouring agent (i.e. a flavourant) is included to enhance the flavour of the liquid composition, preferably when the liquid composition is a beverage. Preferably, pineapple and passion fruit are used to flavour the liquid composition, preferably when the liquid composition is a beverage.

Uses

The skin care formulation according to the present invention may be used in a method for treating breakouts and/or acne, preferably treating cystic acne. As used herein all methods of treatment are cosmetic, non-therapeutic methods.

Accordingly, the present invention provides a method of treating acne comprising administering an effective amount of a skin care formulation according to the present invention to a subject in need thereof. The present invention also provides a method of treating acne comprising administering an effective amount of a skin care formulation according to the present invention to a subject in need thereof wherein the formulation is in the form of a liquid composition and a solid composition. Preferably, the liquid composition and the solid composition are administered concurrently or sequentially. Preferably, the solid composition is administered immediately before or after the liquid composition (preferably as a beverage) has been administered (i.e. less than about 5 minutes, preferably less than about 2 minutes, more preferably less than about 1 minute before or after the liquid composition).

Preferably, the solid composition is administered as one or more capsules, preferably two capsules. Accordingly, the method of treating acne according to the present invention preferably comprises administering one or more capsules comprising an effective amount of the solid composition of the skin care formulation according to the present invention and an effective amount of the liquid composition of the skin care formulation according to the present invention to a subject in need thereof. As will be understood, the skin care formulation is a food supplement.

Preferably, the effective amount of the liquid composition is from about 50 to about 300 mL/day, preferably from about 70 to about 250 mL/day, more preferably from about 100 to about 200 mL/day and the effective amount of the solid composition is from about 250 to about 3000 mg/per day, preferably from about 500 to about 2000 mg/day, more preferably from about 750 to about 1500 mg/day.

Preferably, the treatment is administered daily, preferably for at least about 30 days, preferably for at least about 60 days, more preferably for at least about 90 days. Advantageously, after completing treatment with the present invention, users exhibit increased skin hydration, fewer breakouts, clearer looking skin, reduction in blemishes, reduced inflammation, more balanced oil production, increases skin suppleness and healthier looking skin. Accordingly, the present invention is effective in the treatment of acne.

The present invention also provides a skin care product comprising the solid composition described hereinabove and the liquid composition described hereinabove. The skin care product may be used in a method for treating breakouts or acne, preferably treatment of cystic acne.

Advantageously, providing the skin care product according to the present invention as two parts, i.e. a solid composition for oral administration (preferably as a capsule) and a liquid composition for oral administration (preferably as a beverage), allows all of the ingredients in the product to be absorbed by the body at the rate and concentration required for efficacy. Thus, the skin care product according to the present invention is able to target the multiple specific issues associated with acne described hereinabove rather than only one. Conversely, if the skin care product according to the present invention comprised tablets alone (i.e. without the liquid composition) multiple tablets would be required. Furthermore, in tablet form the ingredients that are present in the liquid composition according to the present invention would not be absorbed at the correct rate and concentration, which are both essential for optimum efficacy of the skin care product.

The skin care formulation according to the present invention may further comprise additional optional components. For example, one or more vitamins, minerals, additives, antioxidants, carbohydrate sources, amino acids, trace elements, hyaluronic acid, glucosamine, fruit/vegetable juices, preservatives (such as potassium sorbate), emulsifiers/hydrocolloids, oils, carbonation components and the like.

The present invention will now be described further with reference to the following examples.

### EXAMPLES

A solid composition is prepared by combining the following ingredients in a conventional manner. All of the ingredients are used as powders.

| Ingredient | Amount (mg) |
|---|---|
| Red Reishi Mushroom extract | 400 |
| Saw Palmetto | 200 |
| Agnus Castus Extract | 200 |
| Lactobacillus | 125.2 |
| Indole-3-Carbinol | 100 |
| Broccoli Extract (5%) | 25 |

The resulting composition (also a powder) is then formulated into 2 capsules. Specifically, the composition is mixed with an appropriate amount of a flow improvement agent (such as magnesium stearate). One half of a hydroxypropyl methyl cellulose and titanium dioxide capsule is then filled with the resulting mixture in a capsule filling machine and the capsule is then sealed with the other half.

A liquid composition is prepared by combining the following ingredients in a conventional manner. The ingredients are used either as an aqueous solution or as microsolids suspended in aqueous solution.

| Ingredient | Amount (mg/15 mL) |
|---|---|
| Milk thistle extract (80%) | 650 |
| Vitamin C (calcium ascorbate) | 500 |
| Methyl sulfonyl methane | 500 |
| Calcareous Marine Algae (Lithothamnion) | 401.6 |
| N-Acetyl-L-Cysteine | 400 |
| Calendula Flower Extract | 250 |
| Magnesium Citrate | 200 |
| Omega-3 DHA Algal Powder | 200 |
| Inositol | 100 |
| Pyridoxine Hydrochloride (vitamin B6) | 20 |
| Niacinamide (vitamin D3) | 20 |
| Zinc Gluconate | 20 |
| Thiamine Hydrochloride (vitamin B1) | 10 |
| Calcium D Pantothenate (vitamin B5) | 10 |
| Retinal Acetate (vitamin A) | 9.23 |
| Riboflavin (vitamin B2) | 6 |
| Folic Acid (vitamin B9) | 0.4 |
| Anhydrous Sodium Selenite | 0.1 |
| Biotin (vitamin B7) | 0.075 |
| Chromium Chloride Hexahydrate | 0.035 |
| Cobalamin (vitamin B12) | 0.02 |

The liquid composition prepared above was then diluted with 150 mL of water and shaken to prepare a beverage.

### Example 2

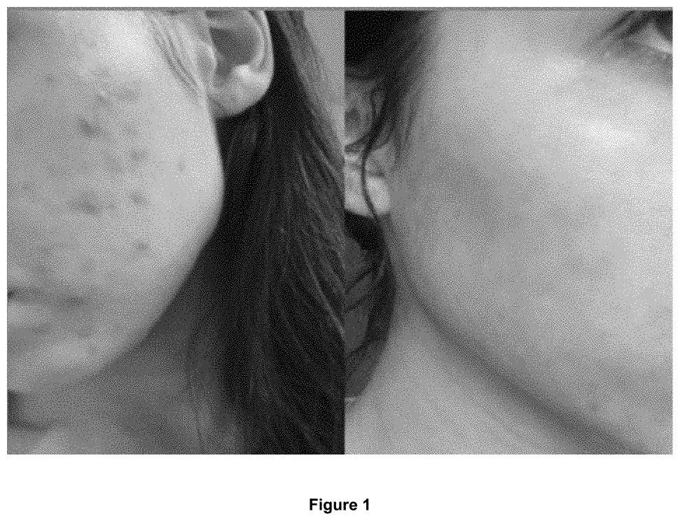

Individuals affected by acne were administered daily with two capsules and one beverage prepared in Example 1 over a period of 30 days and the results from one individual are shown in FIG. 1. Specifically, after 30 days of treatment there was a significant reduction in the appearance of breakouts and users reported that the skin was more balanced, less inflamed, clearer and healthier looking.

### Example 3

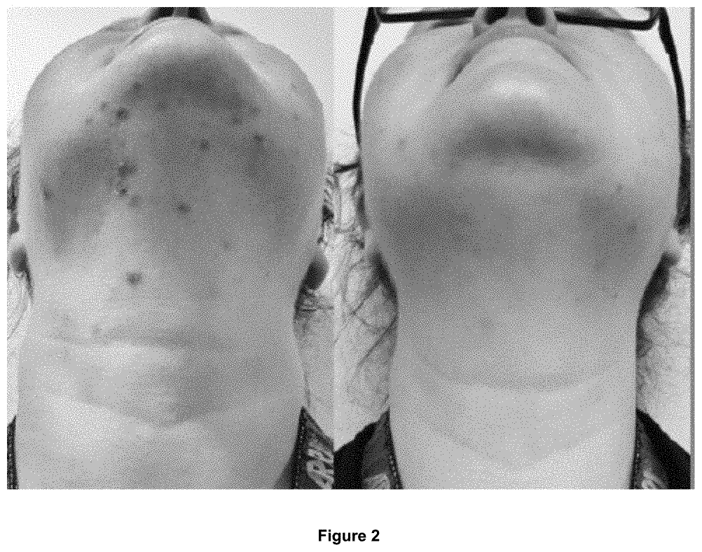

Individuals affected by acne were administered daily with two capsules and one beverage prepared in Example 1 over a period of 90 days and the results from one individual are shown in FIG. 2. Specifically, after 90 days of treatment there was a significant reduction in the appearance of breakouts and users reported that the skin was more balanced, less inflamed, clearer and healthier looking.

### Example 4

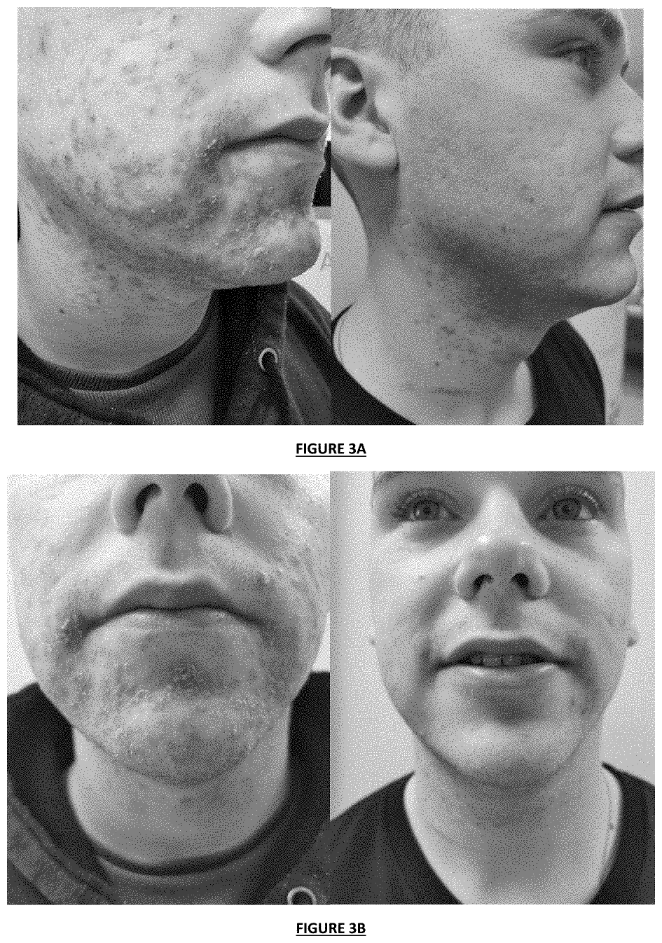

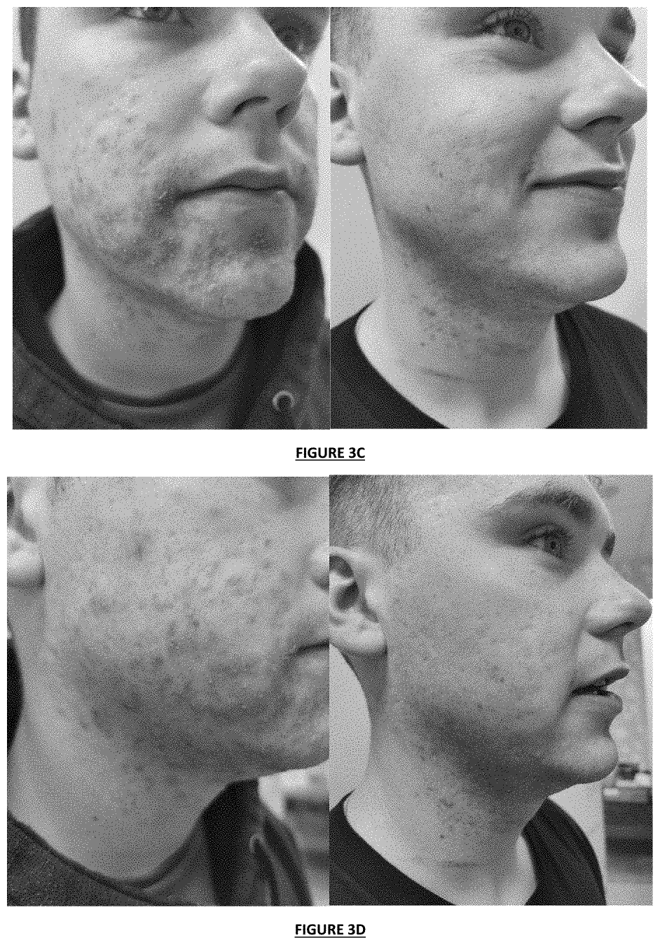

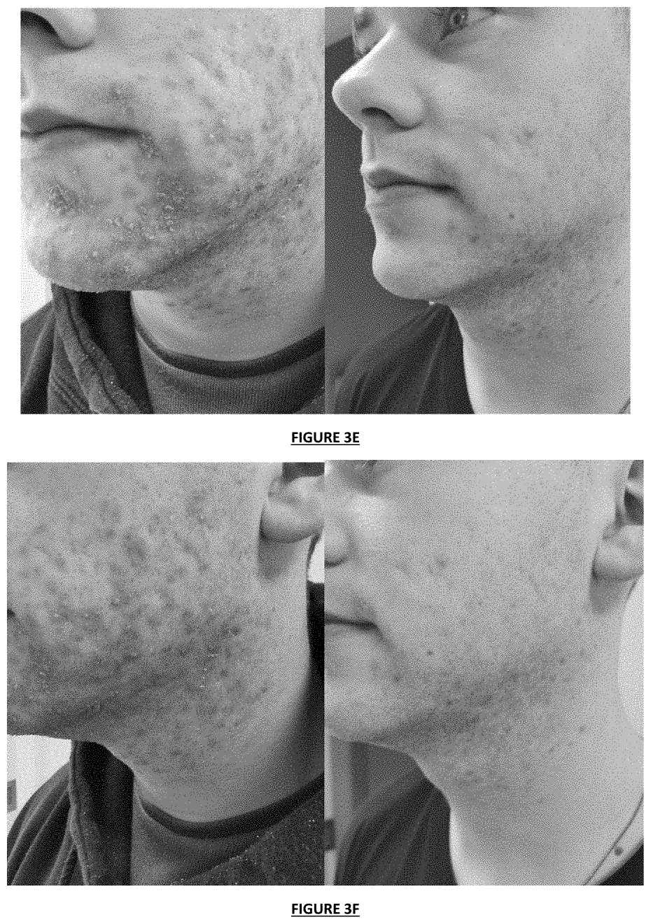

An individual affected by severe acne was administered daily with two capsules and one beverage prepared in Example 1 over a period of 30 days and the results are shown in FIGS. 3A-F. Specifically, after 30 days of treatment there was a significant reduction in the appearance of breakouts and the skin was more balanced, less inflamed, clearer and healthier looking. Treatment will be continued for a further 60 days (90 days total) to achieve full remission.

It should be understood that the present invention has been described only by way of example and that modifications of the formulation may be made within the scope of the accompanying claims.

1. A skin care formulation comprising:
one or more compound(s) that are capable of reducing estrogen concentration in a subject;
one or more compound(s) that are capable of reducing dihydrotestosterone concentration in a subject; and
one or more compound(s) that are capable of reducing insulin concentration in a subject.

2. The skin care formulation according to claim 1 wherein the one or more compound(s) capable of reducing estrogen concentration is selected from indole-3-carbinol, a broccoli extract, an agnus castus extract, magnesium or a salt thereof, and combinations thereof.

3. The skin care formulation according to claim 1 wherein the one or more compound(s) capable of reducing estrogen concentration is a combination of indole-3-carbinol, a broccoli extract and an agnus castus extract.

4. The skin care formulation according to claim 1 wherein the one or more compound(s) capable of reducing estrogen concentration is magnesium or a salt thereof.

5. The skin care formulation according to claim 1 wherein the one or more compound(s) capable of reducing estrogen concentration is a combination of indole-3-carbinol, a broccoli extract, an agnus castus extract and magnesium citrate

6. The skin care formulation according to claim 1 wherein the one or more compound(s) that are capable of reducing dihydrotestosterone concentration is selected from saw palmetto, red reishi mushroom extract, zinc or a salt thereof, and combinations thereof.

7. The skin care formulation according to claim 1 wherein the one or more compound(s) capable of reducing dihydrotestosterone concentration is zinc or a salt thereof.

8. The skin care formulation according to claim 1 wherein the one or more compound(s) that are capable of reducing dihydrotestosterone concentration is a combination of saw palmetto and red reishi mushroom extract.

9. The skin care formulation according to claim 1 wherein the one or more compound(s) that are capable of reducing dihydrotestosterone concentration is a combination of saw palmetto, red reishi mushroom extract and zinc gluconate.

10. The skin care formulation according to claim 1 wherein the one or more compound(s) that are capable of reducing insulin concentration is chromium or a salt thereof.

11. The skin care formulation according to claim 1 further comprising a digestive aid.

12. The skin care formulation according to claim 11 wherein the digestive aid is a probiotic.

13. The skin care formulation according to claim 12 wherein the probiotic is lactobacillus.

14. The skin care formulation according to claim 1 further comprising at least one further active ingredient selected from an omega-3 fatty acid, vitamin A or a derivative thereof, a milk thistle extract, methyl sulphonyl methane (MSM), and combinations thereof.

15. The skin care formulation according claim 1 wherein the formulation is in the form of a solid composition and a liquid composition.

16. The skin care formulation according to claim 15 wherein the solid composition is a capsule.

17. The skin care formulation according to claims 15 wherein the indole-3-carbinol, broccoli extract and agnus castus extract are present in the solid composition.

18. The skin care formulation according to claim 15 wherein the indole-3-carbinol is present in an amount of from about 1 to about 20 wt % of the solid composition.

19. The skin care formulation according to claim 15 wherein the broccoli extract is present in an amount of from about 0.5 to about 10 wt % of the solid composition.

20. The skin care formulation according to claim 15 wherein the agnus castus extract is present in an amount of from about 5 to about 35 wt % of the solid composition.

21. The skin care formulation according to claim 15 wherein saw palmetto and red reishi extract are present in the solid composition.

22. The skin care formulation according to claim 15 wherein the saw palmetto is present in an amount of from about 5 to about 35 wt % of the solid composition.

23. The skin care formulation according to claim 15 wherein the red reishi mushroom extract is present in an amount of from about 10 to about 60 wt % of the solid composition.

24. The skin care formulation according to claim 15 wherein the lactobacillus is present in the solid composition.

25. The skin care formulation according to claim 15 wherein the lactobacillus is present in an amount of from about 1 to about 25 wt % of the solid composition.

26. The skin care formulation according to claim 15 wherein the magnesium or salt thereof, zinc or salt thereof and chromium or salt thereof are present in the liquid composition.

27. The skin care formulation according to claim 15 wherein the magnesium or salt thereof is present in an amount of from about 50 to about 500 mg/15 mL of the liquid composition.

28. The skin care formulation according to claim 15 wherein the zinc or salt thereof is present in an amount of 5 to about 50 mg/15 mL of the liquid composition.

29. The skin care formulation according to claim 15 wherein the chromium or salt thereof is present in an amount of 5 to about 100 μg/15 mL of the liquid composition.

30. The skin care formulation according to claim 15 wherein the omega-3 fatty acid, vitamin A or a derivative thereof, milk thistle extract, methyl sulphonyl methane (MSM), or combinations thereof are in the liquid composition.

31. The skin care formulation according to claim 15 wherein the liquid composition further comprises a liquid diluent.

32. The skin care formulation according to claim 31 wherein the liquid diluent content is at least about 80% by weight of the liquid composition.

33. The skin care formulation according to claim 15 wherein the liquid composition is a beverage.

34. A method of treating acne comprising administering an effective amount of a skin care formulation according to claim 1 to a subject in need thereof.

35. The method of claim 34 wherein the formulation is in the form of a solid composition and a liquid composition according to claim 15 and wherein the method comprises administering the solid composition and liquid composition concurrently or sequentially.

36. The method of claim 35 wherein the effective amount of the liquid composition is from about 50 to about 300 mL/day and the effective amount of the solid composition is from about 250 to about 3000 mg/per day.

37. The method of claim 35 wherein the solid composition is administered in less than about 5 minutes before or after the liquid composition is administered.

38. The method of claim 34 wherein treatment is administered daily for at least about 30 days.
